(19) United States
(12) Patent Application Publication
(10) Pub. No.: US 2018/0228932 A1
(43) Pub. Date: Aug. 16, 2018

(54) COMPREHENSIVE ODOR ELIMINATOR SYSTEM

(71) Applicants: Rhonda M Tiller, Evans, GA (US); Thomas T. Tiller, Evans, GA (US)

(72) Inventors: Rhonda M Tiller, Evans, GA (US); Thomas T. Tiller, Evans, GA (US)

(21) Appl. No.: 15/095,100

(22) Filed: Apr. 10, 2016

(51) Int. Cl. A61L 9/16; B01D 53/04

(52) U.S. Cl. CPC A61L 9/16; A61L 9/014; B01D 53/0415

(57) ABSTRACT

Field of the Invention/Idea:

The present invention/idea is the brain trust of Rhonda M. Tiller. She received this concept in a dream of how to eliminate odors associated with garbage containers, cans, and other odor producing items.

Description of the Related Market:

We recognize that some companies have devised sprays, clip-on, and insert bags for reduced odor control; but these products simply mask the odor. There is a real need to fill this void in the marketplace for odor elimination.

Summary:

1. The first usage is shown to the commercial side of this invention/idea and will be introduced to that segment of the market which already exists as a new concept for customers in order to set this project apart from its competition.

2. The second usage will be packaged and marketed to the domestic side for use in the garbage containers designed for home or office places of conveniences.

### FIELD OF THE INVENTION/IDEA

The present invention/idea is the brain trust of Rhonda M. Tiller. She received this concept in a dream of how to eliminate odors associated with garbage containers, cans, and other odor producing items.

### DESCRIPTION OF THE RELATED MARKET

We recognize that some companies have devised sprays, clip-on, and insert bags for reduced odor control; but these products simply mask the odor. There is a real need to fill this void in the marketplace for odor elimination.

### SUMMARY

1. The first usage is shown to the commercial side of this invention/idea and will be introduced to that segment of the market which already exists as a new concept for customers in order to set this project apart from its competition.

2. The second usage will be packaged and marketed to the domestic side for use in the garbage containers designed for home or office places of conveniences.

### DETAIL DESCRIPTION

The idea for this project was the brain trust of my wife, Rhonda M. Tiller back in November 2012. She had for years thought about how she could eliminate odors associated with the garbage cans, containers and the kitchen trash cans. This idea centers on the creation of an industrial and residential type refillable insert to be replaced monthly with refills of the chemically designed odor eliminator package source designed for the odor elimination system. The uniqueness of this idea has not been fully presented in the context of the eliminating of odors found in trash containers and garbage cans, portal job-johnnies, free standing dumpsters and other similar odor causing containers. Moreover, it is ideal for pet areas such as litter boxes and pet sleeping areas; to demonstrate a list for other extended uses.

Our idea and patent rights covers several untapped areas which makes this idea unique in that we have focused in on an odor elimination system of garbage and trash containers odor control which has far reaching impact on the domestic and foreign markets; commercial and home usage for a wide impact on both private and commercial distribution.

This idea is uniquely designed to be marketed for residential and industrial use for its intended purpose of eliminating odor in garbage and trash containers and/or where other places where offensive odors may occur. This might extend to restrooms and portal job Johnnies. Our odor eliminator system is a new, complete idea which covers the scope of all types of trash and garbage containers in use; both in the home, and; in industrial settings that's used by large commercial suppliers.

Our application for this provisional patent will explain the element that has not been fully obtained before by any other applicants. This idea of a comprehensive odor eliminator system for the elimination of offensive odor that occurs in trash and garbage container has not been applied for in this comprehensive manner before. This is an old problem that many have talked about but this idea to present it in this form of a patent application request is uniquely the idea of its author. All rights to this odor eliminator system belong to its author, Rhonda M. Tiller. Our product is to be marketed both internationally and domestically to

### BRIEF DESCRIPTION OF THE DRAWINGS

FIG. 1 illustrates a perspective view of the straight model passive product dispenser with the permeable mesh product cartridge inside, its typical use condition.

FIG. 2 illustrates an exploded perspective view of the straight model passive product dispenser and permeable mesh product cartridge, from FIG. 1, showing the relationship of the two individual components.

FIG. 3 illustrates an exploded perspective view of the straight model passive product dispenser and permeable mesh product cartridge, from FIG. 1, showing the relationship of the two individual components.

FIG. 4 shows the front view of the container.

FIG. 5 shows the side view of the container.

FIG. 6 shows the back view of the container with section lines for cut-away diagrams, FIGS. 7 and 8.

FIG. 7 shows a side cut-away (vertical section) along the section lines 7-7 from FIG. 6.

FIG. 8 shows a top cut-away (horizontal section plan) along the section lines 8-8 from FIG. 6.

### DETAILED DESCRIPTION OF THE DRAWINGS

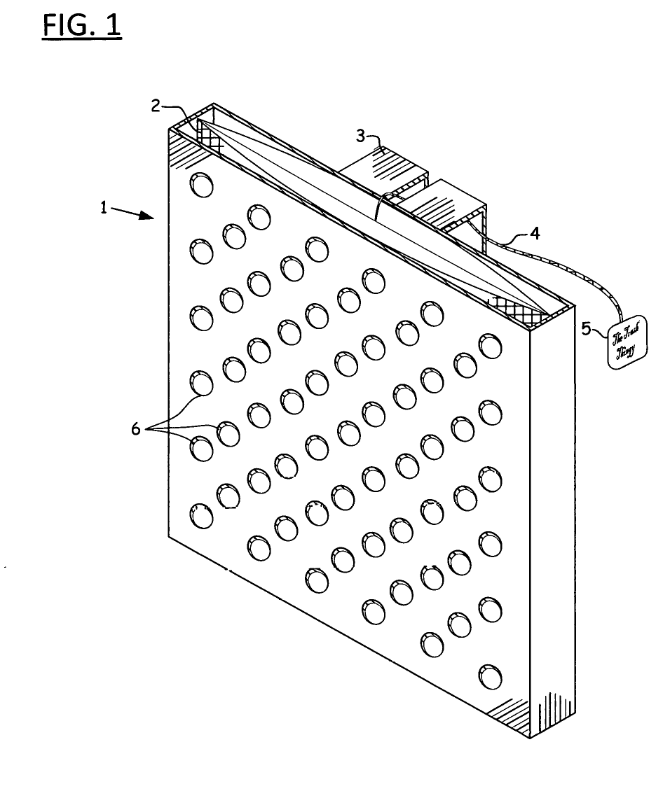

The dispenser illustrated in FIG. 1 represents the most typical configuration of the straight model passive dispenser. Shown are the passive dispenser 1, of plastic or metal construction, designed to be attached or hang on the edge of a household or industrial waste receptacle or any container with odor-producing material. It is therefore equipped with two fixed extension hooks 3 designed to grip to the side of the waste receptacle. The passive dispenser is provided with several holes 6 designed to effectively dispense the odor elimination product into the area being identified for the invention's purpose. The hole pattern is the most typical of Several hole patterns possible. The odor elimination product is contained in a permeable mesh product cartridge 2 that is attached with a cord or string 4 and paper product tag 5, similar to those found on a standard teabag, to enable easy and sanitary removal of spent product cartridges.

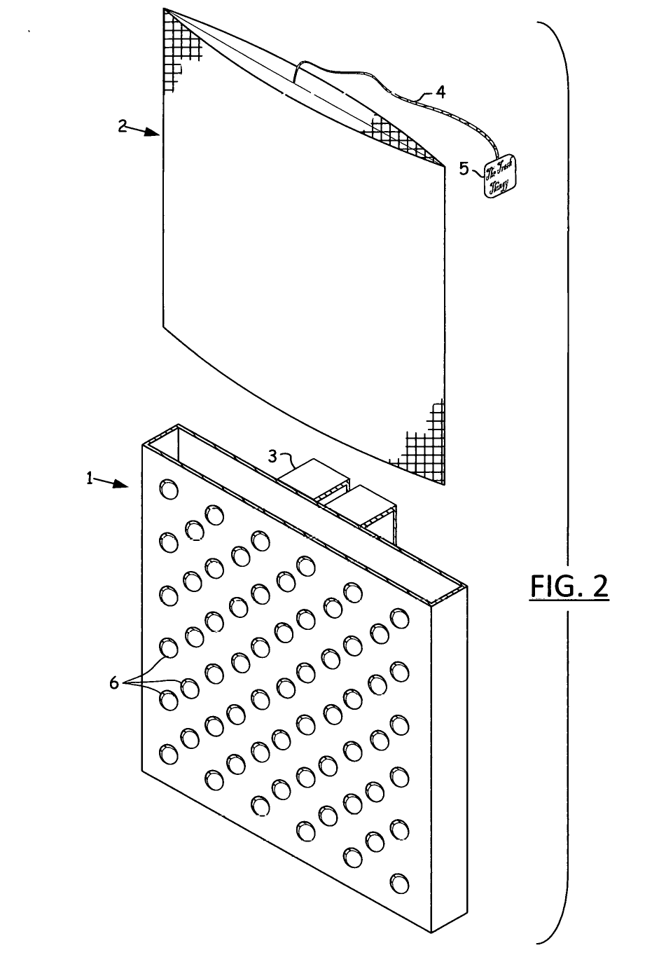

FIG. 2 illustrates an exploded perspective view of the straight model passive product dispenser and permeable mesh product cartridge, from FIG. 1, showing the relationship of the two individual components. Shown are the passive dispenser 1, of plastic or metal construction, designed to be attached or hang on the edge of a household or industrial waste receptacle or any container with odor-producing material. It is therefore equipped with two fixed extension hooks 3 designed to grip to the side of the waste receptacle. The passive dispenser is provided with several holes 6 designed to effectively dispense the odor elimination product into the area being identified for the invention's purpose. The odor elimination product is contained in a permeable mesh product cartridge 2 that is attached with a cord or string 4 and paper product tag 5, similar to those found on a standard tea bag, to enable easy and sanitary removal of spent product cartridges.

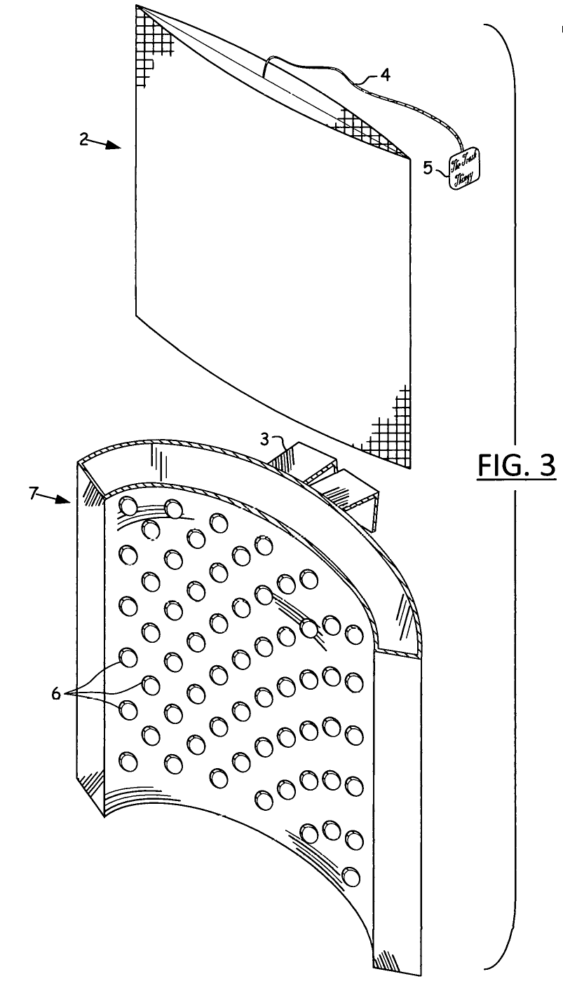

FIG. 3 illustrates an exploded perspective view of the curved model passive product dispenser 7 and permeable mesh product cartridge 2 showing the relationship of the two individual components. This model is identical to the straight model product dispenser 1 with the exception that the curved model product dispenser 7 is designed to fit round waste receptacles. The permeable mesh product cartridge 2 is designed to be flexible so that it can be inserted into either the straight model dispenser 1 or the curved model dispenser 7. FIG. 3 shows the curved model passive dispenser 7, of plastic or metal construction, designed to be attached or hang on the edge of a household or industrial waste receptacle or any container with odor-producing material. It is therefore equipped with two fixed extension hooks 3 designed to grip to the side of the waste receptacle. The passive dispenser is provided with several holes 6 designed to effectively dispense the odor elimination product into the area being identified for the invention's purpose. The odor elimination product is contained in a permeable mesh product cartridge 2 that is attached with a cord or string 4 and paper product tag 5, similar to those found on a standard teabag, to enable easy and sanitary removal of spent product cartridges.

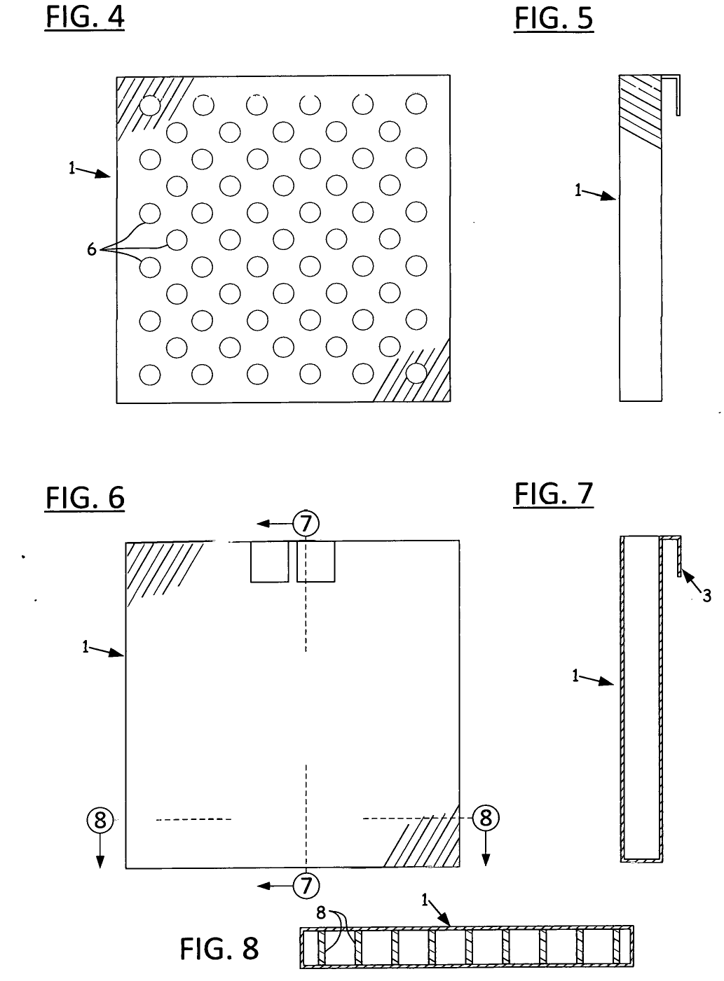

FIG. 4 depicts the external front face view of the straight model passive product dispenser 1 with holes 6 in the most typical pattern for effective dispensing of the product.

FIG. 5 represents an exterior side view of the straight model passive product dispenser 1 with one of the two fixed extension hooks 3 depicted.

FIG. 6 illustrates an exterior back view of the straight model passive product dispenser 1 with the two fixed extension hooks depicted.

FIG. 7 represents a cross sectional side view of the straight model passive produce dispenser 1, taken along lines 7-7 of FIG. 6.

FIG. 8 illustrates a cross sectional plan view of the straight model passive produce dispenser 1, taken along lines 8-8 of FIG. 6. Depicted in this figure are the bottom slats of the passive product dispenser. Except for the slats, the bottom of the passive product dispenser is open to prevent the buildup of trash, or other materials. The slats are also designed to enhance delivery of the odor elimination product into the area being identified for the invention's purpose. major distributors that will be identified, to develop and market this idea in this free market enterprise system.

The odor eliminators used in this process contains constituents similar or such as Carbon, Baking Soda, Coffee and a Combine secret solution. Nevertheless, the usage of this odor eliminator will be copy written and registered for US Patent protection to exclude competition for the statutory period of time. It is anticipated that there will be several proposed usages of the system.

Whereas, the information discussed within this document is confidential and of a proprietary nature which is to be maintained in the strictest confidence. Any breach of this agreement will result in irreparable inquiry to the Tiller family for which damages and other legal remedies will be inadequate. In seeking enforcement of any of these obligations, the Tillers will be entitled to preliminary and permanent injunctive rights and other equitable relief to prevent thief, discontinue and/or restrain the breach of this Agreement in this provisional context during the statutory period.

1. This idea is designed to be marketed for residential and industrial use for its intended purpose of eliminating odor in garbage and trash containers;
Eliminating odors in other places where these offensive odors may occur;
This idea may extend to restrooms and portal job-Johnnies.
This idea is formulated with our secret odor fighting solution which empowers our comprehensive odor eliminator system for the elimination of offensive odor that occurs in trash and garbage containers.
It is further noted that different sizes will be developed to accommodate customer requirements and demands for our comprehensive system.
The Teabag filter contains the odor eliminator product. It is packaged in twelve (12) packages per box. The expected life span for each is one month; and is inserted in the holder on a monthly basis.
The Holder can be purchased as a kit or separately. It is made out of plastic with holes in a certain pattern for emission into the trash containers.
